(19) United States
(12) Patent Application Publication
(10) Pub. No.: US 2023/0061168 A1
(43) Pub. Date: Mar. 2, 2023

(54) Annealing of Discrete Sections of a Reinforcement Layer to Modulate Stiffness of a Catheter

(71) Applicant: DePuy Synthes Products, Inc., Raynham, MA (US)

(72) Inventors: Andres RUIZ, Miami, FL (US); Jorge POSADA, JR., Miami, FL (US)

(21) Appl. No.: 17/446,297

(22) Filed: Aug. 29, 2021

(51) Int. Cl. A61M 25/00; A61M 25/01

(52) U.S. Cl. CPC A61M 25/005; A61M 25/0108; A61M 25/0045; A61M 25/0012

(57) ABSTRACT

A catheter including an inner liner, a reinforcement layer disposed about the inner liner and having a distal edge separated a predetermined axial distance in a proximal direction from the distal end of the inner liner. The reinforcement layer having a discrete annealed section with an altered crystalline structure having modified (e.g., increased) stiffness relative to non-annealed sections of the reinforcement layer. A marker band is positioned over or adjacent to the distal edge of the reinforcement layer. An outer jacket is disposed about an interim assembled structure including the inner liner, the reinforcement layer, and the marker band to form an assembled structure. During manufacture, heat is applied to reflow together individual components of the assembled structure producing an integral composite catheter shaft.

### BACKGROUND OF THE INVENTION

### Field of the Invention

The present invention relates to a catheter used during a medical procedure. For instance, the present invention is applicable to a catheter highly trackable (i.e., the ease by which the catheter follows the guide wire) through tortuous vasculature during an intraluminal, minimally invasive, endovascular medical procedure. The present inventive catheter includes a reinforcement layer a localized or discrete section of which is annealed altering its crystalline structure to modulate (e.g., increase) the stiffness, as desired.

### Description of Related Art

Catheters are widely used in various medical treatments and procedures, for example, intracranial ischemic stroke treatment. Medical treatment procedures often require high trackability or navigation of the catheter through tortuous pathways (e.g., Ophthalmic artery, brachial artery, etc.) without damaging the tissue. It is desirable to provide a highly trackable catheter with increased stiffness without diminishing softness and flexibility of the distal end allowing navigation through tortuous vasculature to a target site in the body without damaging the tissue.

The present invention is a highly trackable catheter with modulated (e.g., increased) stiffness, as desired, along a discrete annealed section thereof while maintaining the softness and flexibility of the distal end able to navigate tortuous pathways without damaging the tissue.

### SUMMARY OF THE INVENTION

An aspect of the present invention is directed to a highly trackable catheter with modulated (e.g., increased) stiffness, as desired, along a discrete annealed section thereof while maintaining the softness and flexibility of the distal end able to navigate tortuous pathways without damaging the tissue.

Another aspect of the present invention is directed to a catheter including an inner liner and a reinforcement layer disposed about the inner liner, wherein the reinforcement layer has a distal edge separated a predetermined axial distance in a proximal direction from the distal end of the inner liner. The reinforcement layer having a discrete annealed section with an altered crystalline structure of modified (e.g., increased) stiffness relative to non-annealed sections of the reinforcement layer. A marker band is positioned over or adjacent to the distal edge of the reinforcement layer. An outer jacket is disposed about an interim structure comprising the inner liner, the reinforcement layer, and the marker band assembled together. During manufacture, heat is applied to the assembled structure causing the individual components (e.g., inner liner, reinforcement layer, marker band and outer jacket) to reflow together producing an integral composite catheter shaft.

Yet another aspect of the present invention relates to a method for manufacture of the catheter described in the preceding paragraph. An inner liner is placed over a mandrel, the inner liner having a proximal end and an opposite distal end. Then, a reinforcement layer is placed about the inner liner so that a distal edge of the reinforcement layer is separated a predetermined distance in a proximal direction from the distal end of the inner liner leaving a distal section of the inner liner exposed. Next, a discrete section of the reinforcement layer is annealed altering its crystalline structure to modify (e.g., increase) stiffness relative to non-annealed sections of the reinforcement layer. A marker band is placed over or immediately adjacent the distal edge of the reinforcement layer. An outer jacket is placed about an interim structure including the inner liner, the reinforcement layer, and the marker band to form an assembled structure. Then heat is applied to reflow together individual components of the assembled structure producing an integral composite catheter shaft.

### BRIEF DESCRIPTION OF THE DRAWING

The foregoing and other features of the present invention will be more readily apparent from the following detailed description and drawings of illustrative of the invention wherein like reference numbers refer to similar elements throughout the several views and in which:

FIG. 1A is a side view depicting the distal end of an exemplary catheter in accordance with the present invention including a uniform (i.e., without any transition) reinforcement layer (e.g., braided layer) a discrete section of which is annealed to modulate (e.g., increase) stiffness, as desired;

FIG. 1B is a partial cut away view of distal end of the exemplary catheter of FIG. 1A illustrating the three different layers (e.g., inner liner; reinforcement layer (e.g., braided layer); outer jacket) formed about a cylindrical mandrel;

FIG. 2 is a side view depicting the distal end of an exemplary catheter in accordance with the present invention including a non-uniform (i.e., having a transition) reinforcement layer (e.g., braided layer) and an outer jacket; wherein a discrete annealed section of the reinforcement layer includes the transition; and

FIG. 3 is a side view depicting the distal end of an exemplary catheter in accordance with the present invention having a uniform (i.e., without or free of any transition) reinforcement layer (e.g., braided layer) a discrete section of which is annealed to modulate (e.g., increase) the stiffness, as desired, and an outer jacket comprising two different polymer layers arranged side-by-side abutting one another along an anticipated interface; wherein the annealed section of the braided layer spans the anticipated interface between the two different polymer layers comprising the outer jacket.

### DETAILED DESCRIPTION OF THE INVENTION

The terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist.

The present inventive intraluminal endovascular catheter comprises multiple layers including, at least, the following: an inner liner (e.g., a lubricious material such as Poly tetrafluoroethylene (PTFE)); a reinforcement layer (e.g., radial crisscross or lattice braiding made of biocompatible metal wires; or a coil formed from a biocompatible metal wire); and an outer jacket (e.g., a polymer such as Polyurethane, Polyethylene, Polyether block amide (PIMA)). The distal end/tip of the inner liner (representing the distal end/tip of the catheter) extends further in a distal direction than the distal edge of the reinforcement layer (i.e., the distal edge of the reinforcement layer terminates proximally of the distal tip/end of the inner liner leaving a distal section of the inner liner exposed) providing the desirable flexibility to the catheter while navigating the tortuous vasculature (e.g., Ophthalmic artery, brachial artery, etc.) to a target site (e.g., in the brain, peripheral vasculature, etc.) without damaging the tissue. Despite these advantages, such flexibility in combination with a relatively large diameter size hampers the trackability of the catheter through tortuous vasculature to the target site when force is applied to the proximal end (pushing in a distal direction). The prevent inventive catheter is highly trackable as a result of modifying/modulating the stiffness (e.g., increasing), as desired, of one or more localized discrete sections of the metal reinforcement layer (e.g., interwoven braided or coil) via annealing without diminishing the flexibility of the distal tip/end of the catheter shaft and the benefits therefrom. Annealing process herein involves heating (e.g., using a laser) a discrete or localized section of the metal wires comprising the reinforcement layer (e.g., interwoven braid or coil) changing its crystalline structure to achieve the desired stiffness. A marker band (e.g., radiopaque marker) is placed over or immediately adjacent to the distal edge of the reinforcement layer. An outer jacket is place about those assembled layers (including the inner liner, the reinforcement layer, the marker band) forming an assembled structure the individual layers of which are reflowed together (via the application of heat) producing an integral structure composite catheter shaft.

Exemplary embodiments or configurations of the present inventive catheter in which the stiffness is modified (increased) along one or more discrete annealed sections of the reinforcement layer are described in detail below. The different embodiments discussed herein represent non-limiting variations in one or more of those basic layers (e.g., inner liner, reinforcement layer, and/or outer jacket) comprising the composite catheter shaft.

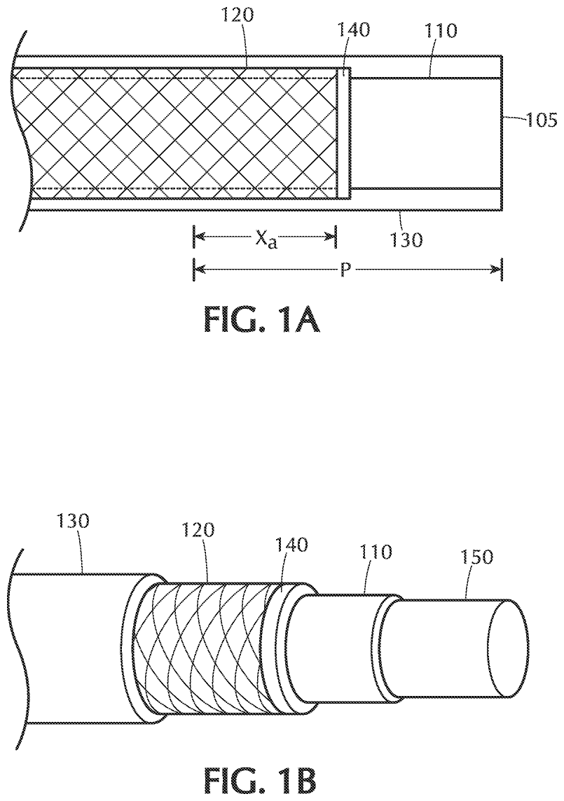

FIG. 1A is a side view of the distal end of the present inventive catheter 100 including an inner liner (e.g., PTFE liner) 110; a metal reinforcement layer 120 (e.g., radial crisscross braiding of biocompatible metal wires (e.g., stainless steel) that is uniform (i.e., non-transitioning or unchanging) in characteristics or properties (e.g., picks per inch (PPI) of the braid); and an outer jacket 130 comprising a polymer material, as shown in FIG. 1B. As previously mentioned, the distal non-reinforced portion of the catheter 100 comprising the exposed (non-reinforced) inner liner 110 extending from the distal edge/side of the reinforcement layer 120 to the distal/end tip 105 is flexible able to navigate through the tortuous vasculature to the target site without damaging the tissue. The stiffness of the metal reinforcement layer 120 along a discrete section (Xa) of the catheter is modulated or modified (e.g., increased) by annealing. Specifically, the discrete section (Xa) of the metal wire lattice comprising the reinforcement layer 120 is heated to alter its crystalline structure to achieve the desired stiffness. The number of discrete annealed section(s) (Xa) and the axial length of each section may be selected, as desired. In the case of multiple discrete annealed sections (Xa), the axial length of each section may, but need not necessarily, be the same. The annealed section (Xa) may be located with its proximal side/edge at a position (p), wherein p is in a range from 0 cm<p≤approximately 300 cm in an axial direction starting from the distal end/tip 105 of the catheter. In a distal direction, the distal edge of the braiding layer 120 terminates prior to the distal end/tip 105 and the annealing section (Xa) of the braided layer 120 is present only on the proximal side/face of the marker band 140 maintaining the flexibility of the remaining non-reinforced (exposed) distal section from the distal edge/side of the marker band 140 and extending in a distal direction including the distal end/tip 105 able to navigate the tortuous vasculature without damaging the tissue.

By way of example, in FIG. 1A the proximal side/edge of the annealed section (Xa) may be located at an axial distance (p) of approximately 20 cm in a proximal direction from the distal end/tip 105 of the intraluminal endovascular catheter; and the longitudinal/axial length of the annealed section (Xa) spans approximately 4 cm in a distal direction (i.e., the distal side/edge of the annealed section (Xa) is located approximately 16 cm in a proximal direction from the distal end/tip 105).

FIG. 1B is a cutaway view depicting the arrangement of the various layers disposed about a mandrel 150 during assembly of the catheter of FIG. 1A. During manufacture the inner liner 110 (e.g., PTFE layer or tube) is positioned about the straight (uniform diameter) cylindrical mandrel 150. Next, the uniform (i.e., non-transitioning or unchanging in characteristics or properties) braided layer 120 is placed about the inner liner 110. In this regard, the mandrel 150 may be woven/braided over (e.g., wound about the outer surface of the mandrel) or the braid may be produced on a separate mandrel and the finished product then slid onto the mandrel 150. A discrete annealed section (Xa) of the distal portion of the braided layer 120 is subject to annealing (i.e., heating to change/alter/modify/modulate the crystalline structure to achieve the desired stiffness). The marker band 140 (e.g., radiopaque material) is placed over or positioned immediately adjacent to a distal edge of the reinforcement layer. Thereafter, the assembled catheter shaft (including the reinforced layer (a discrete portion of which represents the annealed section), the marker band, and the exposed distal portion of the inner liner extending to the distal end/tip) from the proximal end/tip to the opposite distal end/tip is encased in an outer jacket made of a polymer material. The multiple assembled layers are then reflowed together and the sacrificial mandrel is withdrawn (slid out) resulting in the integral composite catheter shaft with an axial/longitudinal lumen defined therethrough formed by the mandrel.

In accordance with this first inventive catheter 100 the annealed section (Xa) of the braided layer 120 may be selected, as desired, so long as the proximal edge/side of the annealed section (Xa) is at a position (p), wherein p is in a range from approximately 0 cm<p≤300 cm, as measured in an axial direction starting from the distal end/tip 105 of the intraluminal endovascular catheter 100.

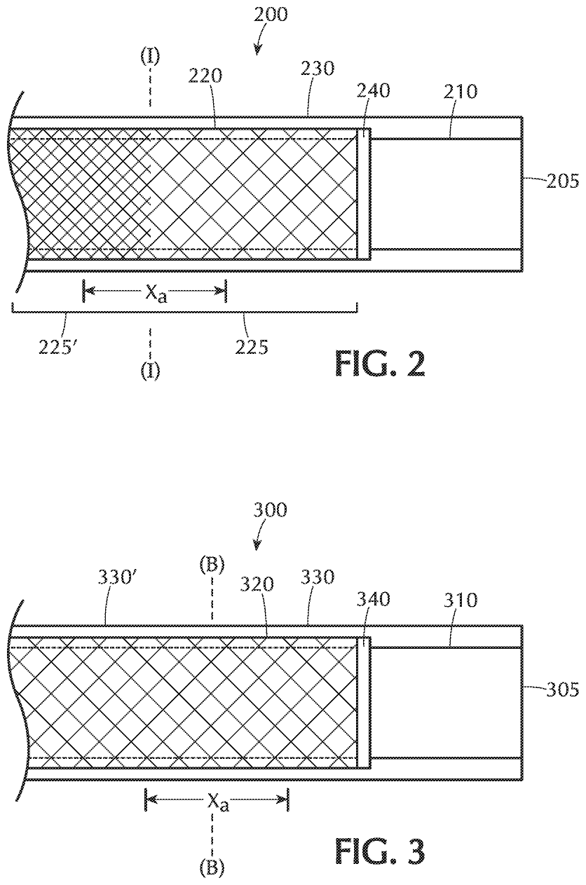

A second embodiment is depicted in the side view of FIG. 2 which differs from that of FIG. 1A in that the reinforcement layer 220 is non-uniform, i.e., there is a transition/change/variation in the reinforcement layer in one or more parameters such as pic count/pitch or shape of the wire (e.g., flat vs. round). In FIG. 2 the reinforcement layer is a radial crisscross braiding of metal wires having at least one braid/pitch transition (e.g., at least one variation of the pic count or pitch (i.e., the number of strand crossovers per inch, or other lineal measure), typically represented by the number of pics per inch (PPI) of the braid). Variable flexibility over the axial length of the braid may be achieved by varying the pic count/pitch (e.g., PPI) and/or shape of the wire. Metal density/wire count changes flexibility. Specifically, the higher the density/pic count the stiffer the structure whereas the lower the density/pic count the more flexible (softer) the structure in relation thereto.

In the illustrated example, braided layer 220 comprises a single interface (I) representing a transition or variation in characteristic or properties along its axial length. For instance, a first portion 225′ of the braided layer between the proximal side/edge and the interface (I) has a first greater pic count (stiffer) relative to that of a second portion 225 of the braided layer between the interface (I) and the distal edge/side having a second lesser pic count (softer). In this way, the first portion of the braid 225′ having the first higher pic count is desirably stiffer relative to the second portion of the braid 225 having the second lower pic count which is softer.

Similar to that of the embodiment in FIG. 1B, the various layers are disposed about a mandrel during assembly of the catheter of FIG. 2. During manufacture an inner liner 210 (e.g., PTFE layer or tube) is positioned about a straight (uniform diameter) cylindrical mandrel 250. The non-uniform (having at least one transition in property or characteristic at an interface) braided layer 220 is positioned/wrapped about the inner liner 210. Specifically, the reinforcement layer may be braided/wrapped directly about the mandrel 250 or produced separately and slid into position onto the mandrel 250. In the example illustrated in FIG. 2, braided layer 220 has a single transition in pic count from a first pic count region 225′ to a second pic count region 225 at an interface (“I”) between the two regions. By way of example, 90 ppi is provided in the second pic count region 225 (closest to the distal edge), whereas 120 ppi is set in the first pic count region 225′ (closest to the proximal edge). Values for pic count of each region may be selected, as desired, depending on the desired properties or characteristics of each region of the reinforcement layer so long as the wire count/density differs between the two regions. Annealed section (Xa) of the braided layer 220 subject to annealing (to change the crystalline structure to achieve the desired stiffness) spans, extends, incorporates, includes, coincides with the transition interface (I). In the example, the annealed section (Xa) has a proximal side/face/edge that is a distance (p) approximately 20 cm from the distal end/tip 205 and the annealed section (Xa) of the braided layer 220 is approximately 4 cm in length in an axial direction. In an axial direction, a midpoint of the annealed section (Xa) of the braided layer 220 and transition interface (I) are aligned with one another so that starting from the transition interface (I) the annealed section (Xa) extends approximately 2 cm in opposing directions (proximal and distal directions). It is noted that the transition interface (I) and midpoint of the annealed section (Xa) need not be aligned with one another so that the axial length of the annealed section in opposing axial directions from the transition interface (I) differs. Marker band 250 (e.g., radiopaque marker) is placed immediately adjacent to or assembled over the distal edge/side of the braided layer 220 securing the free ends of the interwoven wires in place against the inner liner 210. Thereafter, the interim assembled catheter shaft (representing the braided layer (including the annealed section), the marker band, and the exposed (non-reinforced) distal portion of the inner liner 210 extending to the distal end/tip 205) from the proximal end to the opposite distal end is encased in an outer jacket 230 made of a polymer material (e.g., a single polymer material or different polymer materials that are co-extruded or continuously extruded). Lastly, the assembled various layers are reflowed together by applying heat to produce the integral composite catheter shaft. Thus, with this second inventive catheter 200 the annealed section (Xa) of the braided layer 220 may be selected, as desired, to meet the following conditions: (i) the proximal edge/side of the annealed section (Xa) is at a position (p), wherein 0 cm<p≤300 cm, as measured in an axial direction starting from the distal end/tip 205 of the catheter 200; and (ii) the annealed section (Xa) incorporates, spans, includes, or covers the transition interface (I) of the braided layer 220.

Yet a third configuration of the present inventive catheter 300 represented in FIG. 3 has a uniform reinforcement layer 320 (i.e., non-transitioning or unchanging in characteristics or properties (e.g., picks per inch (PPI) of the braid/pitch and/or shape of the wire)); and an outer jacket (330, 330′) comprising two different polymer layers arranged side-by-side one after the other (in series) in an axial direction defining a boundary (“B”) therebetween where the two layers abut (physically contact) one another without overlap. During manufacture of the catheter 300, the two different polymer materials comprising the outer jacket are applied after annealing of the discrete annealed section (Xa) of the reinforcement layer 320, hence prior to the two different outer polymer layers (330, 330′) being applied, the boundary (“B”) therebetween is thus referred to as “expected” or “anticipated.” That discrete section (Xa) of the reinforcement layer 320 that is subject to annealing incorporates, spans, includes or coincides with the “expected” or “anticipated” boundary (“B”) between the to be applied two different polymer layers (330, 330′) comprising the outer jacket. In an exemplary embodiment, the different polymer layers may be the same polymer material differing in hardness. For example, the annealed section (Xa) of the braided layer 320 is approximately 4 cm in axial length while the two different polymer materials arranged axially in series includes a first outer polymer layer 330′ (e.g., urethane with durometer 90A) and a second outer polymer layer 330 (e.g., urethane with durometer 70A) different from that of the first out polymer layer. Still referring to the example, the location of the “expected” or “anticipated” boundary (“B”) between the to be applied two different polymer layers comprising the outer jacket is aligned with the midpoint in an axial direction of the annealed section (Xa) of the braided layer 320.

Accordingly, following the example the discrete annealed section (Xa) will extend approximately 2 cm in length in opposing axial directions (proximal and distal) starting from the “expected” or “anticipated” boundary (“B”) between the to be applied polymer materials (330, 330′) comprising the outer jacket. It is noted that the “expected”/“anticipated” boundary (“B”) and midpoint of the annealed section (Xa) need not be aligned with one another so that the length of the annealed section in opposing axial directions starting from the boundary (“B”) differs. Thus, with this third inventive catheter 300 the annealed section (Xa) of the reinforcement layer 320 may be selected, as desired, to satisfy the following conditions: (i) the proximal edge/side of the annealed section (Xa) is at a position (p), wherein 0<p<300 cm, as measured in an axial direction starting from the distal end/tip 305 of the catheter 300; and (ii) the discrete annealed section (Xa) incorporates, spans, includes, or coincides with the “anticipated”/“expected” boundary (“B”) between the two different polymer layers comprising the outer jacket arranged axially in series when applied following annealing of the discrete section of the braided layer. Such polymer transition in the outer jacket allows force to be more efficiently transferred as the catheter traverses through the anatomy.

It is possible for more than one configuration to be combined together. For instance, the catheter may have a reinforcement layer with a single transition interface (as represented in FIG. 2) and an outer jacket comprising two polymer layers arranged in an axial direction side-by-side one after the other, i.e., in series, abutting one another along a boundary (as shown in FIG. 3). In such combination the discrete annealed section of the reinforced layer would both include the single transition interface as well as the “expected”/“anticipated” boundary between the two polymer material layers comprising the outer jacket. Transitions in the reinforcement layer and/or the outer jacket coinciding with annealed discrete sections of the reinforcement layer contribute to the trackability and durability of the catheter. Also, the illustrative examples in the drawings depict the present inventive catheter having a braided layer as the reinforcement layer but in any of the embodiments or configurations the reinforcement layer may be a coil. It is also noted that the illustrative examples shown in the figures and described in detail include only a single transition interface (I) in the braided layer and/or a single boundary (B) in outer j acket, but more than one (I) and/or (B) is contemplated. The present inventive catheter is suitable for use during an endovascular procedure but is applicable for use in other intraluminal medical procedures.

Thus, while there have been shown, described, and pointed out fundamental novel features of the invention as applied to a preferred embodiment thereof, it will be understood that various omissions, substitutions, and changes in the form and details of the systems/devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A catheter comprising:
an inner liner having a proximal end and an opposite distal end;
a reinforcement layer disposed about the inner liner and having a distal edge separated a predetermined axial distance in a proximal direction from the distal end of the inner liner; the reinforcement layer having a discrete annealed section with an altered crystalline structure having modified stiffness relative to non-annealed sections of the reinforcement layer;
a marker band positioned over or adjacent to the distal edge of the reinforcement layer; and
an outer jacket disposed about an interim structure comprising the inner liner, the reinforcement layer, and the marker band forming an assembled structure individual components of which are reflowable together forming an integral composite catheter shaft.

2. The catheter in accordance with claim 1, wherein a proximal face of the discrete annealed section is located at an axial position (p), wherein 0 cm<p≤approximately 300 cm in a proximal direction starting from the distal end of the inner liner.

3. The catheter in accordance with claim 1, wherein the reinforcement layer is a braided layer of interwoven metal wires or a coil formed from a metal wire.

4. The catheter in accordance with claim 3, wherein the reinforcement layer has at least one transition interface in which at least one property of the reinforcement layer changes; and the discrete annealed section includes the at least one transition interface.

5. The catheter in accordance with claim 4, wherein the at least one transition interface is a change in pic count and/or change in shape of the wire forming the braided layer or coil.

6. The catheter in accordance with claim 1, wherein the outer jacket comprises different material layers arranged side-by-side one after the other in series in an axial direction, wherein the different material layers abut one another along an anticipated boundary; and the discrete annealed section of the reinforcement layer coincides with the anticipated boundary.

7. The catheter in accordance with claim 6, wherein the different material layers are different polymer materials or a single polymer material having different levels of hardness.

8. The catheter in accordance with claim 1, wherein the reinforcement layer has more than one discrete annealed section.

9. The catheter in accordance with claim 1, wherein the modified stiffness of the discrete annealed section has increased stiffness relative to the non-annealed sections of the reinforcement layer.

10. A method for producing a catheter, the method comprising the steps of:
placing an inner liner over a mandrel, the inner liner having a proximal end and an opposite distal end;
placing a reinforcement layer about the inner liner so that a distal edge of the reinforcement layer is separated a predetermined distance in a proximal direction from the distal end of the inner liner leaving a distal section of the inner liner exposed;
annealing a discrete section of the reinforcement layer altering its crystalline structure to modify stiffness relative to non-annealed sections of the reinforcement layer;
placing a marker band over or immediately adjacent the distal edge of the reinforcement layer; and
placing an outer jacket about an interim structure including the inner liner, the reinforcement layer, and the marker band to form an assembled structure; and
applying heat to reflow together individual components of the assembled structure producing an integral composite catheter shaft.

11. The method in accordance with claim 10, wherein a proximal face of the discrete annealed section is located at an axial position (p), wherein 0 cm<p≤approximately 300 cm in a proximal direction starting from the distal end of the inner liner.

12. The method in accordance with claim 10, wherein the reinforcement layer is a braided layer of interwoven metal wires or a coil formed from a metal wire.

13. The method in accordance with claim 12, wherein the reinforcement layer has at least one transition interface in which at least one property of the reinforcement layer changes; and the discrete annealed section includes the at least one transition interface.

14. The method in accordance with claim 13, wherein the at least one transition interface is a change in pic count and/or change in shape of the wire forming the braided layer or coil.

15. The method in accordance with claim 10, wherein the outer jacket comprises different material layers arranged side-by-side one after the other in series in an axial direction, wherein the different material layers abut one another along an anticipated boundary; and the discrete annealed section of the reinforcement layer coincides with the anticipated boundary.

16. The method in accordance with claim 15, wherein the different material layers are different polymer materials or a single polymer material differing in levels of hardness.

17. The method in accordance with claim 10, wherein the reinforcement layer has more than one discrete annealed section.

18. The method in accordance with claim 10, wherein the modified stiffness of the annealed discrete section has increased stiffness relative to the non-annealed sections of the reinforcement layer.
